(19) United States
(12) Patent Application Publication
(10) Pub. No.: US 2022/0125844 A1
(43) Pub. Date: Apr. 28, 2022

(54) OPIOID ANTAGONISTS FOR USE IN PATIENTS USING CHIMERIC ANTIGEN RECEPTOR T AND NATURAL KILLER (NK) CELL THERAPY

(71) Applicant: The Johns Hopkins University, Baltimore, MD (US)

(72) Inventors: Dermot Maher, Washington, DC (US); Nicola Heller, Baltimore, MD (US)

(21) Appl. No.: 17/508,673

(22) Filed: Oct. 22, 2021

Related U.S. Application Data
(60) Provisional application No. 63/104,673, filed on Oct. 23, 2020.

(51) Int. Cl. A61K 35/17; A61K 45/06; A61P 35/00

(52) U.S. Cl. CPC A61K 35/17; A61P 35/00; A61K 45/06

(57) ABSTRACT

Compositions and their use for treating a subject undergoing treatment with chimeric antigen receptor (CAR)-T cells and/or CAR-natural killer (NK) cells comprising administering to the subject one or more opioid antagonists in combination with the CAR-T cells and/or CAR-NK cells are disclosed. The disclosed compositions and methods prevent or attenuates the inhibitory effect of the one or more opioids on the ability of the CAR-T cells and/or CAR-NK cells to induce apoptosis in a tumor cell.

### CROSS-REFERENCE TO RELATED APPLICATIONS

This application claims the benefit of U.S. Provisional Application No. 63/104,673 filed Oct. 23, 2020, the contents of which is herein incorporated by reference in their entirety.

### BACKGROUND

Primary human natural killer (NK) cell cytotoxicity is reduced in vitro if cells are preincubated with clinically-used opioids (e.g., morphine, methadone, and the like) and opioid receptor-specific agonists (e.g., DAMGO, U-50488, and the like). This effect can be prevented by incubation with an opioid antagonist (e.g., naloxone) prior to treatment with an opioid agonist. Retrospective studies have demonstrated that delayed recurrence of cancer following surgery occurs with a decreased incidence if patients received either fewer opioids or an opioid-reducing anesthetic, such as regional anesthesia.

### SUMMARY

In some aspects, the presently disclosed subject matter provides a method for treating a subject undergoing treatment with chimeric antigen receptor (CAR)-T cells and/or CAR-natural killer (NK) cells, the method comprising administering to the subject one or more opioid antagonists, including peripherally-restricted antagonists, in combination with the CAR-T cells and/or CAR-NK cells.

In some aspects, the one or more opioid antagonists are selected from the group consisting of peripherally-restricted opioid antagonists and/or centrally-active opioid antagonists.

In particular aspects, the one or more peripherally-restricted opioid antagonists is selected from the group consisting of naloxegol, methylnatrexone, alvimopan, 6β-naltrexol, axelopran, bevenopran, methylsamidorphan, naldemedine, naltrexamine, and combinations and derivatives thereof.

In some aspects, the centrally-active opioid antagonist is selected from the group consisting of naloxone, naltrexone, nalmefene, diprenorphine, nalorphine, nalorphine dinicotinate, levallorphan, samidorphan, nalodeine, and combinations thereof.

In some aspects, the subject is undergoing or has undergone treatment with one or more opioids for pain. In particular aspects, the one or more opioids have an inhibitory effect on an ability of the CAR-T cells and/or the CAR-NK cells to induce apoptosis in a tumor cell. In more particular aspects, the administration of the one or more opioid antagonists prevents or attenuates the inhibitory effect of the one or more opioids on the ability of the CAR-T cells and/or CAR-NK cells to induce apoptosis in a tumor cell.

In some aspects, the one or more opioid antagonists is administered concurrently with, prior to, or after administration of the CAR-T cells and/or CAR-NK cells.

In other aspects, the method further comprises administering one or more additional therapeutic agents, including in some aspects, a chemotherapeutic agent, in combination with the one or more opioid antagonists.

In some aspects, the subject is undergoing or has undergone treatment for cancer. In particular aspects, the cancer comprises a hematological malignancy or a solid tumor.

In more particular aspects, the hematological malignancy is selected from the group consisting of an aggressive, relapsed or refractory non-Hodgkin lymphoma, diffuse large B cell lymphoma (DLBCL), primary mediastinal B-cell lymphoma, high grade B-cell lymphoma, DLBCL resulting from follicular lymphoma, refractory or relapsed mantle cell lymphoma, relapsed or refractory acute lymphoblastic leukemia (ALL) (up to age 25), and relapsed or refractory leukemia and lymphomas expressing CD19-positive tumors.

In certain aspects, the solid tumor is selected from the group consisting of thyroid cancer, liver cancer, pancreatic cancer, brain tumor, breast cancer, ovarian tumor, colorectal cancer, recurrent or refractory B-cell tumor, lung cancer, gastric cancer, advanced gastric adenocarcinoma, pancreatic adenocarcinoma, advanced EGFR-positive solid tumors, advanced glioma, and advanced solid tumor.

In some aspects, the subject has undergone or is undergoing one or more additional treatments for cancer selected from the group consisting of chemotherapy, radiation therapy, chemoradiation therapy, surgery, an additional immunotherapy, and combinations thereof.

In some aspects, the treatment with the CAR-T cells comprises one or more CAR-T cell therapies selected from the group consisting of brexucabtagene autoleucel, tisagenlecleucel, and axicabtagene ciloleucel. In other aspects, the CAR-T cell therapy includes genetically-modified lymphocytes, such as innate and adaptive lymphocytes, and other leukocytes.

In some aspects, the CAR-NK cells are derived from a source selected from the group consisting of adult peripheral blood, umbilical cord blood, and induced pluripotent stem cells.

In other aspects, the presently disclosed subject matter provides a method of inhibiting opioid signaling in a T cell or a natural killer (NK) cell, the method comprising altering expression of one or more opioid receptors in the T cell or NK cell, wherein opioid binding to the one or more opioid receptors is disrupted and opioid signaling is inhibited in the T cell or NK cell.

In other aspects, the presently disclosed subject matter provides a composition comprising one or more of CAR-T cells and/or CAR-NK cells and one or more opioid antagonists as disclosed herein.

In some aspects, the composition further comprises one or more opioids.

Certain aspects of the presently disclosed subject matter having been stated hereinabove, which are addressed in whole or in part by the presently disclosed subject matter, other aspects will become evident as the description proceeds when taken in connection with the accompanying Examples and Figure as best described herein below.

### BRIEF DESCRIPTION OF THE FIGURE

Having thus described the presently disclosed subject matter in general terms, reference will now be made to the accompanying Figure, which is not necessarily drawn to scale, and wherein:

FIG. 1 shows the effect of morphine and peripherally restricted opioid antagonists on the ability of primary human NK and CAR-NK cells to induce apoptosis in MV-4-11 cells.

### DETAILED DESCRIPTION

The presently disclosed subject matter now will be described more fully hereinafter with reference to the accompanying Figure, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figure. Therefore, it is to be understood that the presently disclosed subject matter is not to be limited to the specific embodiments disclosed and that modifications and other embodiments are intended to be included within the scope of the appended claims.

### Opioid Antagonists for Use in Patients Using Chimeric Antigen Receptor T and Natural Killer (NK) Cell Therapy

The use of opioid analgesic reduces the function of chimeric antigen receptor (CAR)-T cells and CAR-NK cells. In some embodiments, the presently disclosed subject matter provides for the concurrent use of an opioid antagonist for treating patients undergoing treatment with chimeric antigen receptor (CAR)-T cells and/or CAR-NK cells. Without wishing to be bound to any one particular theory, it is thought that the concurrent use of an opioid antagonist with CAR-T cells and/or CAR-NK cells prevents or attenuates the deleterious effect of an opioid analgesic on the ability of CAR-T cells and/or CAR-NK cells to induce apoptosis in tumor cells.

Accordingly, in some embodiments, the presently disclosed subject matter provides a method for treating a subject undergoing treatment with chimeric antigen receptor (CAR)-T cells and/or CAR-natural killer (NK) cells, the method comprising administering to the subject one or more opioid antagonists, including peripherally restricted antagonists, in combination with the CAR-T cells and/or CAR-NK cells.

The term “chimeric antigen receptor” (“CAR”), as used herein, refers to a recombinant polypeptide construct comprising at least an extracellular antigen binding domain, a transmembrane domain and an intracellular signaling domain. Upon binding to their target (e.g., displayed on a cancer cell), CARs typically modify the immune response of the immune cells on which they are displayed. T cells which express CARs are referred to in the art as “CAR T cells,” “CAR-T cells,” or “CART cells,” while natural killer (NK) cells that express CARs are referred to as “CAR NK cells” or “CAR-NK cells.”

As used herein, an “opioid antagonist” includes a receptor antagonist that acts on one or more opioid receptors, including the μ-opioid receptor (MOR), the κ-opioid receptor (KOR), the δ-opioid receptor (DOR), and combinations thereof. Most opioid antagonists known in the art are μ-opioid receptor antagonists, but many also bind the κ-opioid receptor and/or the δ-opioid receptor.

In some embodiments, the opioid antagonist is a competitive antagonist, which binds to an opioid receptor with a higher affinity than an agonist, but does not activate the receptor. This competitive binding effectively blocks the receptor, thereby preventing the body from responding to an opioid. Naloxone and naltrexone are representative competitive opioid antagonists. Inverse agonists bind the opioid receptor producing an effect in the opposite direction as an agonist. For example, nalmefene is an inverse agonist that is not peripherally restricted.

In some embodiments, the one or more opioid antagonists are selected from the group consisting of peripherally-restricted opioid antagonists and/or centrally-active opioid antagonists.

As used herein, “centrally-active opioid antagonists” refer to opioid receptor antagonists that block one or more of the opioid receptors in the central nervous system and the peripheral nervous system. For example, the competitive antagonism of the central μ-opioid receptors stimulates the respiratory drive, increases alertness, terminates analgesia and euphoria, and causes mydriasis. Representative centrally-active opioid antagonists include, but are not limited to, naloxone, naltrexone, nalmefene, diprenorphine, nalorphine, nalorphine dinicotinate, levallorphan, samidorphan, nalodeine, and combinations thereof. Samidorphan can be both centrally and peripherally acting.

As used herein, “peripherally-restricted opioid antagonists” refer to opioid antagonists that act primarily on physiological systems and components external to the central nervous system, e.g., they do not readily cross the blood-brain barrier in an amount effective to inhibit the central effects of opioids. For example, peripheral opioid antagonists typically exhibit activity with respect to gastrointestinal tissue, while exhibiting reduced or substantially no central nervous system (CNS) activity. Peripheral opioid antagonists are generally μ- and/or κ-opioid antagonists.

In some embodiments, the one or more peripherally-restricted opioid antagonists is selected from the group consisting of naloxegol, methylnatrexone, alvimopan, 6β-naltrexol, axelopran, bevenopran, methylsamidorphan, naldemedine, naltrexamine, and combinations and derivatives thereof.

One of ordinary skill in the art would recognize that some opioid agonists may act as agonists towards one receptor and antagonists toward another receptor and are classified as agonist/antagonists, (also known as mixed or partial agonists). These opioids include, but are not limited to, pentazocine, butorphanol, nalorphine, nalbuphine, buprenorphine, bremazocine, and bezocine. For example, nalorphine is a mixed agonist that is centrally acting. Further, diprenorphine is a partial agonist. Many of the agonist/antagonist group of opioids are agonists at the κ- and δ-opioid receptors and antagonists at μ-opioid receptors.

In some embodiments, the subject is undergoing or has undergone treatment with one or more opioids for pain. In particular embodiments, the one or more opioids is selected from the group consisting of a μ-opioid agonist, a κ-opioid agonist, a δ-opioid agonist, and combinations thereof. In more particular embodiments, the one or more opioids is selected from the group consisting of morphine, methadone, oxycodone, hydrocodone, hydromorphone, tapentadol, oxymorphone, heroin, levorphanol, meperidine, codeine, buprenorphine, loperamide, fentanyl, fentanyl derivatives, meperidine, sufentanil, alfentanil, tramadol, O-desmethyltramadol (ODMT), [D-Ala2, N-Me-Phe4, Gly5-ol-enkephalin (DAMGO), 2-(3,4-dichlorophenyl)-N-methyl-N-[(1R,2R)-2-pyrrolidin-1-ylcyclohexyl]acetamide (U-50488), D-Pen2,D-Pen5]enkephalin (DPDPE), and pharmaceutically acceptable salts thereof. Also included are long-acting, short-acting and abuse deterrent formulations of these drugs. Representative methods of delivering opioid medications include, but are not limited to, oral, transdermal, intravenous, sublingual, transmucosal, rectal, inhaled, insufflated, subcutaneous, intrathecal, epidural, or as part of a regional anesthetic or combination thereof.

In some embodiments, the one or more opioids have an inhibitory effect on an ability of the CAR-T cells and/or the CAR-NK cells to induce apoptosis in a tumor cell. In particular embodiments, the administration of the one or more opioid antagonists prevents or attenuates the inhibitory effect of the one or more opioids on the ability of the CAR-T cells and/or CAR-NK cells to induce apoptosis in a tumor cell.

In some embodiments, the one or more opioid antagonists are administered in an effective amount to prevent or attenuate the inhibitory effect of the one or more opioids. In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, and the like.

As provided hereinabove, the one or more opioid antagonists is administered in combination with the CAR-T cells and/or CAR-NK cells.

The term “combination” is used in its broadest sense and means that a subject is administered at least two agents, more particularly one or more opioid antagonists is administered in combination with the CAR-T cells and/or CAR-NK cells. More particularly, the term “in combination” refers to the concomitant administration of two (or more) active agents for the treatment of, e.g., a single disease state or a single desired physiological and/or pharmacological outcome. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of opioid antagonists described herein can be administered alone or in combination with adjuvants that enhance their stability, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of one or more opioid antagonists in combination with the CAR-T cells and/or CAR-NK cells can be varied so long as the beneficial effects of the combination of these agents are achieved. Accordingly, the phrase “in combination with” refers to the administration of one or more opioid antagonists and the CAR-T cells and/or CAR-NK cells either concurrently, sequentially, or a combination thereof. Therefore, a subject administered a combination of one or more opioid antagonists and the CAR-T cells and/or CAR-NK cells can receive one or more opioid antagonists and the CAR-T cells and/or CAR-NK cells at the same time (i.e., concurrently) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. When administered concurrently, the agents can be administered to the subject as separate pharmaceutical compositions, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of one or more opioid antagonists and the CAR-T cells and/or CAR-NK cells is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} \text{/} {Q}_{A} + {Q}_{b} \text{/} {Q}_{B} = Synergy Index ( SI )$$

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Accordingly, in some embodiments, the one or more opioid antagonists is administered concurrently with administration of the CAR-T cells and/or CAR-NK cells. In other embodiments, the one or more opioid antagonists is administered prior to administration of the CAR-T cells and/or CAR-NK cells. In some embodiments, the one or more opioid antagonists is administered after administration of the CAR-T cells and/or CAR-NK cells.

In some embodiments, the method further comprises administering one or more additional therapeutic agents in combination with the one or more opioid antagonists. In some embodiments, the one or more additional therapeutic agents are selected from the group consisting of an anticancer agent, an antiviral agent, an antiretroviral agent, a protease inhibitor, a nucleoside analog, a nucleotide analog, an anti-infective agent, a hematopoietic stimulating agent, and combinations thereof.

In some embodiments, the anticancer agent comprises a chemotherapeutic agent. In some embodiments, the chemotherapeutic agent is selected from the group consisting of Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

Other anti-cancer drugs include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; 9-dihydrotaxol; dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedap latin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic lycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfm; vinorelbine; vinxaltine; vitaxin; vorozole; zanoterone; zeniplatin; zilascorb; and zinostatin stimalamer.

In some embodiments, the subject is undergoing or has undergone treatment for cancer. In particular embodiments, the cancer comprises a hematological malignancy or a solid tumor. In certain embodiments, the hematological malignancy is selected from the group consisting of an aggressive, relapsed or refractory non-Hodgkin lymphoma, diffuse large B cell lymphoma (DLBCL), primary mediastinal B-cell lymphoma, high grade B-cell lymphoma, DLBCL resulting from follicular lymphoma, refractory or relapsed mantle cell lymphoma, relapsed or refractory acute lymphoblastic leukemia (ALL) (up to age 25), and relapsed or refractory leukemia and lymphomas expressing CD19-positive tumors.

In certain embodiments, the solid tumor (and representative target antigens) can include one or more of the following: (TSHR) thyroid cancer; (CD133) liver cancer, pancreatic cancer, brain tumor, breast cancer, ovarian tumor, and colorectal cancer; (CD19) recurrent or refractory B-cell tumor; (CEA) lung cancer, colorectal cancer, gastric cancer, breast cancer, pancreatic cancer; (Claudin 18.2) advanced gastric adenocarcinoma and pancreatic adenocarcinoma; and (EGFR) advanced EGFR-positive solid tumors, advanced glioma, and advanced solid tumor.

In some embodiments, the subject has undergone or is undergoing one or more additional treatments for cancer selected from the group consisting of chemotherapy, radiation therapy, chemoradiation therapy, surgery, an additional immunotherapy, and combinations thereof.

In some embodiments, the treatment with the CAR-T cells comprises one or more CAR-T cell therapies selected from the group consisting of brexucabtagene autoleucel (Tecartus™), tisagenlecleucel (Kymriah™), and axicabtagene ciloleucel (Yescarta™)

In other embodiments, the CAR-T cell therapy includes genetically-modified lymphocytes, such as innate and adaptive lymphocytes, and other leukocytes.

In some embodiments, the CAR-NK cells are derived from a source selected from the group consisting of adult peripheral blood, umbilical cord blood, and induced pluripotent stein cells.

### Modulation of Opioid Signaling in T cells and NK cells

It will be appreciated that opioid signaling within T cells or NK cells, such as CAR-T cells or CAR-NK cells, may be antagonized or inhibited by disrupting the binding interaction of an opioid and its cognate receptor. Thus, the present disclosure also provides a method of inhibiting opioid signaling in a T cell or a natural killer (NK) cell, which comprises altering expression of one or more opioid receptors in the T cell or NK cell, wherein opioid binding to the one or more opioid receptors is disrupted and opioid signaling is inhibited in the T cell or NK cell. The four major opioid receptor families include opioid receptor μ (OPRM), opioid receptor κ (ORPK), opioid receptor δ (OPRD) and opioid related nociceptin receptor 1 (OPRL; also referred to as “opioid receptor-like orphan receptor”). Opioid receptors exist not only in the nervous system, but also in peripheral organs, such as heart, lungs, liver, gastrointestinal, and reproductive tracts (Feng et al., Curr Drug Targets, 13(2): 230-246 (2012)).

In some embodiments, altering expression of one or more opioid receptors comprises altering a gene that encodes an opioid receptor. “Altering” a gene or DNA sequence refers to modifying at least one physical feature of a wild-type DNA sequence of interest. DNA alterations include, for example, single or double strand DNA breaks, deletion or insertion of one or more nucleotides, and other modifications that affect the structural integrity or nucleotide sequence of the DNA sequence. A gene encoding an opioid receptor may be altered using any suitable method for introducing targeted, sequence-specific changes to a nucleic acid sequence. For example, homologous recombination or gene editing methods may be used to alter an opioid receptor gene. The terms “gene editing” and “genome editing,” as used herein, refer to technologies that allow genetic material to be inserted, removed, or altered at a particular location in the genome. Gene editing technologies that may be used in the context of this disclosure include, but are not limited to, zinc-finger nucleases (ZFNs), transcription activator-like effector nucleases (TALENs), and CRISPR (clustered regularly interspaced short palindromic repeat DNA sequences)/Cas elements. Gene editing technologies are further described in, e.g., Appasani, K. (ed.), Genome Editing and Engineering: From TALENs, ZFNs and CRISPRs to Molecular Surgery, 1st ed., Cambridge University Press (2018); and Hirakawa et al., Biosci Rep. 2020; 40(4): BSR20200127. doi:10.1042/BSR20200127; Hall et al., Overview: Generation of Gene Knockout Mice. Current Protocols in Cell Biology, 44; Wiley-Blackwell. pp. Unit 19.12 19.12., 1-17 (2009); Santiago et al., Proceedings of the National Academy of Sciences. 105 (15): 5809-5814 (2008); Gaj et al., Trends in Biotechnology. 31 (7): 397-405 (2013); Joung, J. K. and Sander, J. D., Nature Reviews Molecular Cell Biology. 14 (1): 49-55 (2013); Ni et al., PLOS ONE. 9 (9): e106718 (2014); and Le, Y. and Sauer, B., Molecular Biotechnology. 17 (3): 269-275 (2001)).

In other embodiments, altering expression of one or more opioid receptors may be achieved using RNA interference (RNAi). The term “RNA interference” refers to a process in which RNA molecules inhibit gene expression or translation by neutralizing targeted mRNA molecules. To achieve an RNAi effect, for example, RNA having a double strand structure containing the same base sequence as that of the target mRNA may be used. Two types of small RNA molecules may induce RNAi: microRNA (miRNA) and small interfering RNA (siRNA). miRNA is a small non-coding RNA molecule (containing about 22 nucleotides) found in plants, animals and some viruses, which silences complementary target sequences by one or more of the following processes: (1) cleavage of the target mRNA strand into two pieces, (2) destabilization of the mRNA through shortening of its poly(A) tail, and (3) less efficient translation of the mRNA into proteins by ribosomes (Bartel D. P., Cell, 136 (2): 215-233 (2009); and Fabian et al., Annual Review of Biochemistry, 79: 351-79 (2010)). siRNA (also known as short interfering RNA or silencing RNA), is a class of double-stranded RNA molecules, typically 20-25 base pairs in length, which silence complementary target sequences by degrading mRNA after transcription, preventing translation (Dana et al., International Journal of Biomedical Science, 13(2):48-57 (2017); and Agrawal et al., Microbiol. Mol. Biol. Rev., 67: 657-685 (2003)). siRNA can also act in RNAi-related pathways as an antiviral mechanism or play a role in the shaping of the chromatin structure of a genome. Any RNA molecule that is capable of silencing gene expression of a target gene may be used in connection with the present disclosure. In some embodiments, the RNA molecule is siRNA. RNA interference is further described in, e.g., Fire et al., Nature. 391 (6669): 806-11 (1998); Pratt, A. J., and MacRae, I. J., The Journal of Biological Chemistry. 284 (27): 17897-901 (2009); Fraser et al., Nature. 408 (6810): 325-30 (2000); Esvelt et al., Nature Methods. 10 (11): 1116-21 (2013); and Sun, N. and Zhao, H., Biotechnology and Bioengineering. 110 (7): 1811-21 (2013).

The T cells or NK cells in which opioid receptor gene expression has been altered may be ex vivo, in vivo, or in vitro. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context.

### Compositions

In some embodiments, the presently disclosed subject matter provides a composition comprising one or more of CAR-T cells and/or CAR-NK cells and one or more opioid antagonists. In some embodiments, the one or more opioid antagonists are selected from the group consisting of peripherally-restricted opioid antagonists and/or centrally-active opioid antagonists. In some embodiments, the one or more peripherally-restricted opioid antagonists is selected from the group consisting of naloxegol, methylnatrexone, alvimopan, 6β-naltrexol, axelopran, bevenopran, methylsamidorphan, naldemedine, and combinations and derivatives thereof.

In some embodiments, the centrally-active opioid antagonist is selected from the group consisting of naloxone, naltrexone, nalmefene, diprenorphine, nalorphine, nalorphine dinicotinate, levallorphan, samidorphan, nalodeine, naltrexamine, and combinations and derivatives thereof.

In some embodiments, the composition further comprises one or more opioids. In some embodiments, the one or more opioids is selected from the group consisting of a μ-opioid agonist, a κ-opioid agonist, a δ-opioid agonist, and combinations thereof.

In some embodiments, the one or more opioids is selected from the group consisting of morphine, methadone, oxycodone, hydrocodone, hydromorphone, tapentadol, oxymorphone, heroin, levorphanol, meperidine, codeine, buprenorphine, loperamide, fentanyl, fentanyl derivatives, meperidine, sufentanil, alfentanil, tramadol, O-desmethyltramadol (ODMT), [D-Ala2, N-Me-Phe4, Gly5-ol]-enkephalin (DAMGO), 2-(3,4-dichlorophenyl)-N-methyl-N-[(1R,2R)-2-pyrrolidin-1-ylcyclohexyl]acetamide (U-50488), D-Pen2,D-PenS]enkephalin (DPDPE), and pharmaceutically acceptable salts thereof.

In some embodiments, the compositions further comprise a pharmaceutically acceptable carrier.

One of skill in the art will recognize that the pharmaceutical compositions include the pharmaceutically acceptable salts of the compounds described above. Pharmaceutically acceptable salts are generally well known to those of ordinary skill in the art and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galacturonic acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra -sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hanks' solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods

### 1.1 Human NK Cell Isolation and Chimeric Antigen Receptor (CAR) Modification

NK cells were isolated from three individual healthy donors. Subsequently, chimeric antigen modification of the cells was conducted. Both the original NK cells and the modified CAR-NK cells from the same three donors were a gift from the department of oncology. RPMI-1640 (Gibco), with 10% foetal bovine serum (Gemini Bio Products, West Sacramento, Calif.) and 100 units of penicillin and streptomycin per mL (Gibco, Gaithersburg, Md.), were used as growth media in subsequent experiments.

### 1.2 Drug Treatments

Stock solutions of morphine and naloxone were obtained from the hospital pharmacy and diluted in PBS. All clinically used medications in this study, including naloxone, were racemic mixtures. Other drugs procured included methylnaltrexone (Cayman Chemical, Ann Arbor, Mich.), naloxegol (Cayman Chemical, Ann Arbor, Mich.), 613-naltrexol (Cayman Chemical, Ann Arbor, Mich.), alvimopan (Cayman Chemical, Ann Arbor, Mich.), naldemedine (Cayman Chemical, Ann Arbor, Mich.), and staurosporine (LKT Laboratories, St. Paul, Minn.).

Effector NK and CAR-NK cells were incubated with opioid antagonists or an equivalent volume of PBS for two hours and then with morphine 25 ng/mL for an additional two hours. All clinically used drugs were used over a concentration range described elsewhere as clinically relevant. Boland and Pockley, 2016.

A two-hour incubation period was chosen as this time period represents a biologically plausible NK cell exposure duration following parenteral administration. Concentrations of drugs tested include: morphine 25 ng/mL, naloxone 100 ng/mL, methylnaltrexone 250 ng/mL, naloxegol 100 ng/mL, Alvimopam 20 ng/mL, 6β-Naltrexol 20 ng/mL, and naldemedine 20 ng/mL.

### 1.3 MV-4-11 Cell Culture and CFSE Staining

MV-4-11, a non-adherent, acute myelogenous leukaemia-derived cell line devoid of MEW I complexes, was chosen as the standardized target tumor cell for the NK and CAR-NK cell apoptosis assay because it is non-adherent and can be analysed by flow cytometry without the need for physical manipulation. Similar assays have been extensively described in NK cell apoptosis assays and extensively used in the inventor's laboratory. Fischer et al., 2002; Aubry et al., 1999; Maher et al, 2019a; Maher et al., 2020. Before using the cells in an apoptosis assay, they were counted them and their viability was checked with 0.4% trypan blue exclusion dye (Gibco). Then, 2×10⁶ of the MV-4-11 cells were labelled with 5-μM carboxyfluorescein succinimidyl ester (CFSE; Thermo Fisher) according to the manufacturer's instructions.

### 1.4 Apoptosis Assay, Annexin V Staining, and Flow Cytometry

2×10⁴ CFSE-labelled MV-4-11 target cells in 50 μL of media were added to each well of a 96-well plate containing 2×10⁴NK or CAR-NK cells. The cells were mixed with gentle pipetting and the plate was placed in an incubator for 30 min. Three wells containing only CF SE-labelled MV-4-11 cells were used as a negative control. As a positive control, three wells that contained untreated NK or CAR-NK cells and CFSE-labelled MV-4-11 cells were included. Finally, as a positive control for apoptosis, three wells of MV-4-11 cells exposed to 100 mM staurosporine, which has been demonstrated to induce apoptosis in AML cells lines, Oliver et al., 2011, were included.

After the 30-min incubation, the cells were stained with annexin V-APC (Biolegend, San Diego, Calif.) according to the manufacturer's instructions and 50 μM propidium iodide (Sigma-Aldrich). Cells were analysed immediately on a CytoFLEX flow cytometer with a 96-well plate reader and CytEXPERT software (version 2.1; Beckman-Coulter, Indianapolis, Ind.). A sample of 1×10⁴ CFSE positively staining cells was collected from each well. The percent of CFSE-gated cells that stained positive for annexin V was determined (i.e., the percent of MV-4-11 cells undergoing apoptosis).

### 1.5 Analysis and Statistics

Results were analysed with Kaluza software, version 2.0 (Beckman-Coulter). Statistical analysis was carried out with Graphpad Prism, version 7.0 (La Jolla, Calif.) and PS Power and Sample Size Calculation, version 3.0 (Nashville, Tenn.).

For each subject, the average of the three positive control wells (CAR-NK or NK cells and MV-4-11 cells without any treatment) was calculated and denoted as “100% effector cell cytolytic efficiency.” Each treatment was carried out in triplicate in 3 subjects for a total of 9 samples per data point. Each subject had both primary human NK cells and modified CAR-NK cells tested. For each treatment condition, the average of the three experimental wells was determined and the ratio of three experimental wells to the average of the three control wells was determined. Given the relatively small sample size, the normality of the data could not be adequately assessed and so nonparametric analyses were conducted. For each treatment, unpaired nonparametric analyses (Kruskall-Wallis) were conducted with a post-hoc Dunn's test in order to compare multiple treatment means to the positive control of untreated NK or CAR-NK and MV-4-11 cells. Two sided hypotheses were tested. A corrected p-value of less than 0.05 was considered statistically significant.

### Example 2

### Results

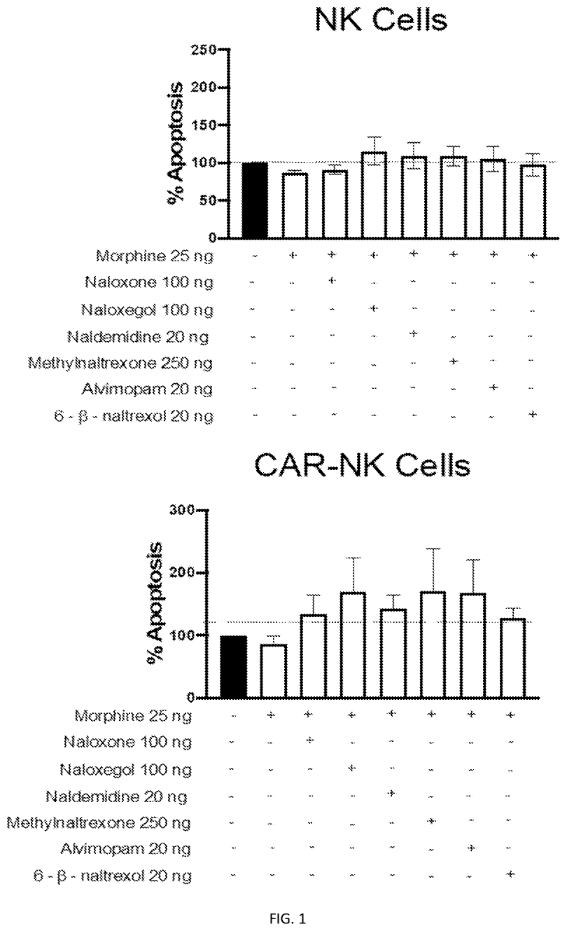

To evaluate the effect of morphine on NK and CAR-NK cell cytolytic function, previously optimized assays were used to measure the induction of apoptosis in tumor target cells. The means and standard deviations are graphically presented in FIG. 1

### Example 3

### Conclusions

For many patients, the clinical course of cancer unfortunately results in the development of cancer pain and surgical pain with the need for pain treatment, often with medications, such as opioids. Portenoy, 2011. Patients also may concurrently have non cancer-associated pain requiring opioid therapy. Until recently, it was believed that opioid analgesics were relatively benign and without a significant impact on long-term oncologic outcomes. Clinical evidence, however, has found an association between the use of high doses of perioperatively administered opioids and greater rates of cancer recurrence, prompting a preclinical investigation of the effects of opioids on natural killer (NK) cells and other cellular components of the innate immune system. Maher et al., 2014; Biki et al., 2008, Exadaktylos, et al., 2006; Maher and White, 2016.

NK cells are primarily involved in surveillance and eradication of tumor cells including those dislodged following surgical manipulation. CAR-NK and CAR-T cells represent a novel mechanism of treating tumors. Treatment with CAR-NK and CAR-T cells, however, is resource and financially intensive. There is concern that patients who receive CAR-NK or CAR-T cell therapy while also treating pain with opioid analgesics will suffer a decrease in the effectiveness of their CAR-T or CAR-NK therapy. Given the significant cost, efforts should be made to preserve the effectiveness of this therapy.

Peripherally-restricted opioid receptor antagonists block the effect of morphine and other opioids at receptors outside the central nervous system (e.g., beyond the blood brain barrier). Peripherally-restricted opioid receptor antagonists were primarily developed and marketed for treatment of opioid-induced constipation. They allow for the patient to have the peripheral effects of opioids to be blocked, while preserving the pain-reducing effects of opioids in the central nervous system. Lymphocytes also are restricted to the periphery.

CAR-T and CAR-NK cells are administered into the bloodstream (e.g., the periphery). In this compartment, they would be fully exposed to the effects of opioids in the periphery. Without wishing to be bound to any one particular theory, it is thought that clinically-relevant concentrations of opioids in the periphery would decrease the ability of CAR-NK cells to induce apoptosis, an early stage of cellular death, in tumor cells. This indicates that there is decreased function of CAR-NK cells and a decreased ability to efficiently induce apoptosis and or necrosis in targeted tumor cells. It also is thought that this decrease in the ability of CAR-NK cells to induce apoptosis could be prevented by the administration of peripherally-restricted opioid antagonists.

It has been previously demonstrated that the cytotoxic functional ability of human NK cells to induce apoptosis in target tumor cells is decreased if the NK cells are first exposed in vitro to a wide variety of clinically-used opioids in a concentration-dependent manner and that this decrease can be reversed with the non-peripherally restricted opioid antagonists, e.g., naloxone. Maher et al., 2019a. In keeping with those findings, the presently disclosed subject matter demonstrates that: (1) similar to primary human NK cells, CAR-NK cells are functionally inhibited by clinically-relevant concentrations of morphine; and (2) peripherally-restricted opioid antagonists can prevent the decrease in function of both primary NK and CAR-NK cells.

To this end, several peripherally-restricted opioid antagonists were tested; several of which are currently available in the U.S. and others have been evaluated for safety in phase I trials, but were not developed for commercial reasons.

The clinical utility for the presently disclosed subject matter is the ability to maximize the effectiveness of CAR-NK and CAR-T cell therapies in patients who frequently require opioid-analgesia for the treatment of cancer-associated pain or other pain with opioids.

### Example 4

### Genetic Manipulation of Opioid Receptors on CAR-T and CAR-NK Cells

The cytotoxic function of primary human NK cells and T cells is decreased after exposure to opioids. This decrease is believed to be due to binding of opioids directly to opioids receptors. This signaling can be decreased or eliminated by several mechanisms involving genetic manipulation of these cells. This modification may or may not occur at the same time as the modification of those cells to express a chimeric antigen receptor (CAR).

Representative examples of genetic modification include, but are not limited to, gene knockdown with RNA interference (RNAi), gene knock out with clustered regularly interspaced short palindromic repeats (CRISPR/Cas9), zinc fingers, homologous recombination or transcription activator-like effector nucleases (TALENs). The knock in of other nonfunctional opioid binding proteins (e.g., a dominant negative) is another method of achieving a similar outcome. The genes of interest include OPRM, ORPK, OPRD and OPRL.

Decreased signaling through these opioid receptors will allow for both native NK and T cells, as well as CAR-T and CAR-NK cells, to less efficiently exert cytotoxic and signalling functions against target tumor cells or other targets.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Boland J W, Pockley A G. Clinically relevant concentrations of opioids for in vitro studies. Journal of opioid management. 2016; 12(5):313-321.

Fischer K, Andreesen R, Mackensen A. An improved flow cytometric assay for the determination of cytotoxic T lymphocyte activity. J Immunol Methods. 2002; 259(1-2):159-169.

Aubry J P, Blaecke A, Lecoanet-Henchoz S, et al. Annexin V used for measuring apoptosis in the early events of cellular cytotoxicity. Cytometry. 1999; 37(3):197-204.

Maher D P, Walia D, Heller N M. Suppression of Human Natural Killer Cells by Different Classes of Opioids. Anesthesia and analgesia. 2019a.

Maher D P, Walia D, Heller N M. Morphine decreases the function of primary human natural killer cells by both TLR4 and opioid receptor signaling. Brain, behavior, and immunity. 2020.

Oliver L, Hue E, Rossignol J, et al. Distinct Roles of Bcl-2 and Bcl-Xl in the Apoptosis of Human Bone Marrow Mesenchymal Stem Cells during Differentiation. PLoS One. 2011; 6(5).

Portenoy R K. Treatment of cancer pain. Lancet. 2011; 377(9784):2236-2247.

Maher D P, Wong W, White P F, et al. Association of increased postoperative opioid administration with non-small-cell lung cancer recurrence: a retrospective analysis. British journal of anaesthesia. 2014; 113 Suppl 1:i88-i94.

Biki B, Mascha E, Moriarty D C, Fitzpatrick J M, Sessler D I, Buggy D J. Anesthetic technique for radical prostatectomy surgery affects cancer recurrence. Anesthesiology. 2008; 109(2):180-187.

Exadaktylos A K, Buggy D J, Moriarty D C, Mascha E, Sessler D I. Can anesthetic technique for primary breast cancer surgery affect recurrence or. Anesthesiology. 2006; 105(4):660-664.

Maher D P, White P F. Proposed mechanisms for association between opioid usage and cancer recurrence after surgery. Journal of clinical anesthesia. 2016; 28:36-40.

Hall, Bradford; Limaye, Advait; Kulkarni, Ashok B. (2009 Sep. 1). Overview: Generation of Gene Knockout Mice. Current Protocols in Cell Biology. 44. Wiley-Blackwell. pp. Unit 19.12 19.12.1-17

Santiago, Yolanda; Chan, Edmond; Liu, Pei-Qi; Orlando, Salvatore; Zhang, Lin; Urnov, Fyodor D.; Holmes, Michael C.; Guschin, Dmitry; Waite, Adam (2008 Apr. 15). “Targeted gene knockout in mammalian cells by using engineered zinc-finger nucleases”. Proceedings of the National Academy of Sciences. 105 (15): 5809-5814

Gaj, Thomas; Gersbach, Charles A.; Barbas, Carlos F. (2013). “ZFN, TALEN, and CRISPR/Cas-based methods for genome engineering”. Trends in Biotechnology. 31 (7): 397-405

Joung, J. Keith; Sander, Jeffry D. (January 2013). “TALENs: a widely applicable technology for targeted genome editing”. Nature Reviews Molecular Cell Biology. 14 (1): 49-55

Ni, Wei; Qiao, Jun; Hu, Shengwei; Zhao, Xinxia; Regouski, Misha; Yang, Min; Polejaeva, Irina A.; Chen, Chuangfu (2014 Sep. 4). “Efficient Gene Knockout in Goats Using CRISPR/Cas9 System”. PLOS ONE. 9 (9): e106718

Le, Yunzheng; Sauer, Brian (2001 Mar. 1). “Conditional gene knockout using cre recombinase”. Molecular Biotechnology. 17 (3): 269-275.

Fire A, Xu S, Montgomery M K, Kostas S A, Driver S E, Mello C C (February 1998). “Potent and specific genetic interference by double-stranded RNA in Caenorhabditis elegans”. Nature. 391 (6669): 806-11

Pratt A J, MacRae I J (July 2009). “The RNA-induced silencing complex: a versatile gene-silencing machine”. The Journal of Biological Chemistry. 284 (27): 17897-901.

Fraser A G, Kamath R S, Zipperlen P, Martinez-Campos M, Sohrmann M, Ahringer J (November 2000). “Functional genomic analysis of C. elegans chromosome I by systematic RNA interference”. Nature. 408 (6810): 325-30.

Esvelt K M, Mali P, Braff J L, Moosburner M, Yaung S J, Church G M (November 2013). “Orthogonal Cas9 proteins for RNA-guided gene regulation and editing” (PDF). Nature Methods. 10 (11): 1116-21.

Sun N, Zhao H (July 2013). “Transcription activator-like effector nucleases (TALENs): a highly efficient and versatile tool for genome editing”. Biotechnology and Bioengineering. 110 (7): 1811-21.

Maher D P, Walia D, Heller N M. Morphine Decreases the Function of Primary Human Natural Killer Cells by Both TLR4 and Opioid Receptor Signaling. Brain Behav Immun. 2020 January;83:298-302.

Maher D P, Walia D, Heller N M. Suppression of Human Natural Killer Cells by Different Classes of Opioids. Anesthesia and analgesia. 2019 May;128(5):1013-1021.

Although the foregoing subject matter has been described in some detail by way of illustration and example for purposes of clarity of understanding, it will be understood by those skilled in the art that certain changes and modifications can be practiced within the scope of the appended claims.

1. A method for treating a subject undergoing treatment with chimeric antigen receptor (CAR)-T cells and/or CAR-natural killer (NK) cells, the method comprising administering to the subject one or more opioid antagonists in combination with the CAR-T cells and/or CAR-NK cells.

2. The method of claim 1, wherein the one or more opioid antagonists are selected from the group consisting of peripherally-restricted opioid antagonists and/or centrally-active opioid antagonists.

3. The method of claim 2, wherein the one or more peripherally-restricted opioid antagonists is selected from the group consisting of naloxegol, methylnatrexone, alvimopan, 6β-naltrexol, axelopran, bevenopran, methylsamidorphan, naldemedine, naltrexamine, and combinations and derivatives thereof and wherein the centrally-active opioid antagonist is selected from the group consisting of naloxone, naltrexone, nalmefene, diprenorphine, nalorphine, nalorphine dinicotinate, levallorphan, samidorphan, nalodeine, and combinations thereof.

4. (canceled)

5. The method of claim 1, wherein the subject is undergoing or has undergone treatment with one or more opioids for pain.

6. The method of claim 5, wherein the one or more opioids is selected from the group consisting of a μ-opioid agonist, a κ-opioid agonist, a δ-opioid agonist, and combinations thereof.

7-8. (canceled)

9. The method of claim 5, wherein the one or more opioids have an inhibitory effect on an ability of the CAR-T cells and/or the CAR-NK cells function including their ability to induce apoptosis in a tumor cell.

10. The method of claim 9, wherein the administration of the one or more opioid antagonists prevents or attenuates the inhibitory effect of the one or more opioids on the ability of the CAR-T cells and/or CAR-NK cells to induce apoptosis in a tumor cell.

11-13. (canceled)

14. The method of claim 1, further comprising administering one or more additional therapeutic agents in combination with the one or more opioid antagonists.

15. The method of claim 13, wherein the one or more additional therapeutic agents are selected from the group consisting of an anticancer agent, an antiviral agent, an antiretroviral agent, a protease inhibitor, a nucleoside analog, a nucleotide analog, an anti-infective agent, a hematopoietic stimulating agent, and combinations thereof.

16. (canceled)

17. The method of claim 1, wherein the subject is undergoing or has undergone treatment for cancer.

18-23. (canceled)

24. A method of inhibiting opioid signaling in a T cell or a natural killer (NK) cell, the method comprising altering expression of one or more opioid receptors in the T cell or NK cell, wherein opioid binding to the one or more opioid receptors is disrupted and opioid signaling is inhibited in the T cell or NK cell.

25. The method of claim 24, wherein altering expression of one or more opioid receptors comprises altering a gene that encodes an opioid receptor.

26. The method of claim 25, wherein altering a gene that encodes an opioid receptor is performed using homologous recombination or a gene editing system.

27. (canceled)

28. The method of claim 24, wherein altering expression of one or more opioid receptors is performed using RNA interference (RNAi).

29. The method of claim 24, wherein the T cell is a CAR-T cell and/or the NK cell is a CAR-NK cell.

30. The method of claim 24, wherein the T cell or the NK cell is in vitro.

31. The method of claim 24, wherein the one or more opioid receptors are selected from opiod receptor μ (OPRM), opioid receptor κ (ORPK), opioid receptor δ (OPRD) and opioid related nociceptin receptor 1 (OPRL).

32. A composition comprising one or more of CAR-T cells and/or CAR-NK cells and one or more opioid antagonists.

33. The composition of claim 32, wherein the one or more opioid antagonists are selected from the group consisting of peripherally-restricted opioid antagonists and/or centrally-active opioid antagonists.

34-35. (canceled)

36. The composition of claim 32, further comprising one or more opioids.

37-39. (canceled)
